(19) United States
(12) Patent Application Publication
(10) Pub. No.: US 2023/0096500 A1
(43) Pub. Date: Mar. 30, 2023

(54) LIGANDS FOR TRANSITION METAL CATALYSTS

(71) Applicant: Rutgers, The State University of New Jersey, New Brunswick, NJ (US)

(72) Inventors: Michal Szostak, Newark, NJ (US); Qun Zhao, Newark, NJ (US); Guangrong Meng, Newark, NJ (US); Guangchen Li, Newark, NJ (US)

(21) Appl. No.: 17/791,673

(22) PCT Filed: Jan. 8, 2021
(86) PCT No.: PCT/US2021/012726

§ 371 Date: Jul. 8, 2022

Related U.S. Application Data
(60) Provisional application No. 62/958,565, filed on Jan. 8, 2020.

(51) Int. Cl. B01J 31/22; B01J 31/18; B01J 31/24; C07D 233/62; C07D 235/02

(52) U.S. Cl. CPC B01J 31/2273; B01J 31/2278; B01J 31/2295; B01J 31/181; B01J 31/2404; C07D 233/62; C07D 235/02; B01J 2531/824; B01J 2531/18; B01J 2531/822; B01J 2231/42

(57) ABSTRACT

Provided herein, in part, is a new class of sterically bulky, easily prepared N-heterocyclic carbene (NHC) ligands of Formula I, or a salt, solvate, geometric isomer, or stereoisomer thereof. The ligands are readily synthetically accessible exploiting the cost-effective, modular alkylation of anilines. The NHC ligands of the present disclosure can be used to prepare effective catalysts with transition metals, including the compound of Formula II, or a salt, solvate, geometric isomer, or stereoisomer thereof. In certain embodiments, the transition metal is Pd.

### CROSS-REFERENCE TO RELATED APPLICATION

This application claims priority to U.S. Provisional Patent Application Ser. No. 62/958,565 entitled “LIGANDS FOR TRANSITION METAL CATALYSTS,” filed Jan. 8, 2020, the disclosure of which is incorporated herein by reference in its entirety.

### STATEMENT REGARDING FEDERALLY SPONSORED RESEARCH

This invention was made with government support under CHE-1650766 awarded by the National Science Foundation and under GM133326 awarded by the National Institutes of Health. The government has certain rights in this invention.

### BACKGROUND

N-heterocyclic carbenes (NHCs) have emerged as tremendously valuable ligands in homogeneous catalysis. The broad application of NHC ligands is a consequence of strong 6-donation of the carbene center and variable steric bulk of wingtip groups that are often not easily attainable using other classes of ligands, including phosphines. Furthermore, through exploiting flexible steric bulk of the wingtips, NHC ligands allow for kinetic stabilization of metals and intermediates at unusual oxidation states, while their well-defined topology has found widespread application in fine-tuning of reactivity at the metal center.

In this context, by far the most important NHC ligand in the field of homogeneous catalysis is bulky IPr (FIG. 1, 1) first reported by Ardengo and Nolan in 1999. While its synthesis from Dipp (Dipp=2,6-diisopropylaniline) is facile, the problem lies in the preparation of Dipp precursor. The preparation of Dipp is severely limited by challenges in controlling the alkylation selectivity, and the most common industrial route exploits lengthy and inflexible route through phenol alkylation.

Therefore, there remains a need for economically and synthetically accessible ligands for transition metals that form efficient and stable catalysts. The present disclosure addresses and solves this need.

### BRIEF SUMMARY OF THE INVENTION

Provided herein are compounds of Formula I, having the structure:

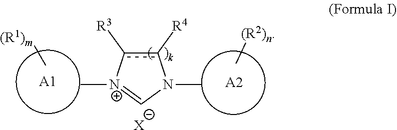

In certain embodiments, [symbol] is a single or double bond. In certain embodiments, A1 and A2 are each independently C₆₋₁₈ aryl or C₆₋₁₈ heteroaryl. In certain embodiments, R¹ and R² are each independently C₁₋₃ alkyl substituted with at least one aryl or heteroaryl, wherein the aryl or heteroaryl in R¹ and R² is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl. In certain embodiments, R³ and R⁴ are each independently hydrogen, optionally substituted C₃₋₁₀ cycloalkyl, optionally substituted aryl, optionally substituted heteroaryl, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), or OC₁₋₁₂ alkyl, wherein the optional substitution independently comprises at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl. In certain embodiments, R³ and R⁴ taken together with the ring to which they are attached are used to form a C₄₋₂₀ cycloalkyl, C₆₋₂₀ aryl, or C₆₋₂₀ heteroaryl, each of which is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl. In certain embodiments, X is a counter anion. In certain embodiments, R is independently at each occurrence hydrogen or C₁₋₁₀ hydrocarbyl (such as, but not limited to, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, and optionally substituted C₁₋₁₀ acyl). In certain embodiments,
k is 1, 2, 3, or 4. In certain embodiments, m is an integer from 0 to 6. In certain embodiments, n is an integer from 0 to 6. In certain embodiments, if A1 and A2 are phenyl, then at least one of m or n is 3, 4, or 5.

Also provided are compounds of Formula II, or a salt thereof:

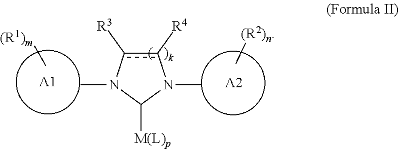

In certain embodiments, M is an element of Group VIII to Group XVI with an atomic weight greater than 25. In certain embodiments, L is a ligand of M, wherein at each occurrence L can be the same or different. In certain embodiments, p is an integer from 0 to 5. In certain embodiments, [symbol] is a single or double bond. In certain embodiments, A1 and A2 are each independently C₆₋₁₀ aryl or C₆₋₁₀ heteroaryl. In certain embodiments, R¹ and R² are each independently C₁₋₃ alkyl substituted with at least one aryl or heteroaryl, wherein the aryl or heteroaryl in R¹ and R² is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl. In certain embodiments, R³ and R⁴ are each independently hydrogen, optionally substituted C₃₋₁₀ cycloalkyl, optionally substituted aryl, optionally substituted heteroaryl, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), or OC₁₋₁₂ alkyl, wherein the optional substitution independently comprises at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl. In certain embodiments, R³ and R⁴ taken together with the ring to which they are attached are used to form a C₄₋₂₀ cycloalkyl, C₆-20 aryl, or C₆₋₂₀ heteroaryl, each of which is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl. In certain embodiments, R is independently at each occurrence hydrogen or C₁₋₁₀ hydrocarbyl (such as, but not limited to, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl). In certain embodiments, k is 1, 2, 3, or 4. In certain embodiments, m is an integer from 0 to 5. In certain embodiments, n is an integer from 0 to 5. In certain embodiments, if A1 and A2 are phenyl, then at least one of m or n is 3, 4, or 5.

### BRIEF DESCRIPTION OF THE FIGURES

The drawings illustrate generally, by way of example, but not by way of limitation, various embodiments of the present application.

FIG. 1 shows a variety if sterically-demanding N-heterocyclic carbenes used in catalysis.

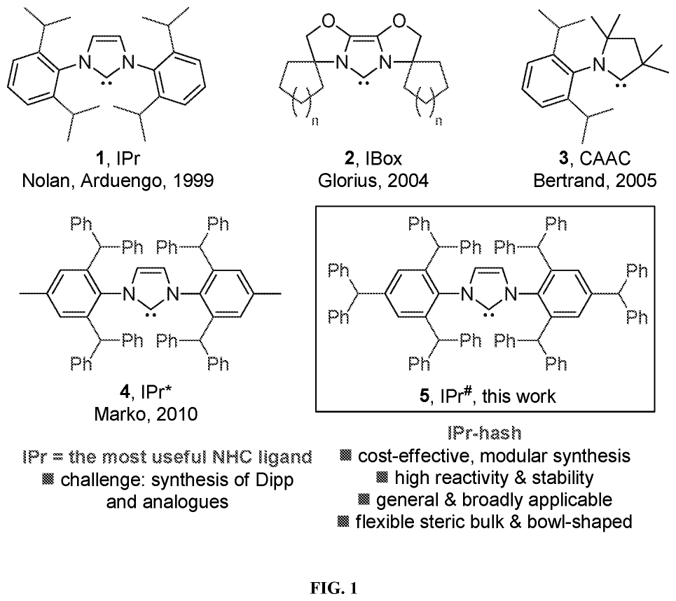

FIG. 2 shows a X-ray crystal structure of complex 9. Two views: front (top); side (bottom). Hydrogen atoms have been omitted for clarity. Selected bond lengths [Å] and angles [° ]: Au-C1, 1.972; Au-Cl,2.2768; C1-N1, 1.356; C3-N1, 1.442(2); C1-Au— Cl,180.0; N1-C1-N1, 104.9; C3-N1-C1, 122.7; N1-C1-Au, 127.5. Note the symmetry across the Cl— Au-C1 axis in 9.

FIG. 3 shows a X-ray crystal structure of complex 13. Two views: front (top); side (bottom). Hydrogen atoms have been omitted for clarity. Selected bond lengths [Å] and angles [° ]: Pd-C1, 2.044(4); Pd-C1, 2.374(1); Pd-C49, 2.121(4); Pd-C50, 2.130(6); Pd-C51, 2.210(7); C1-N1, 1.364(5); C1-N2, 1.368(4); C4-N1, 1.452(4); C58-N2, 1.440(5); C1-Pd-C49, 103.2(2); C1-Pd-C50, 138.5(2); C1-Pd-C51, 169.4(2); C49-Pd-C51, 67.0(2); C1-Pd-C1, 93.7(1); N1-C1-N2, 103.3(3); C4-N1-C1, 124.9(3); C58-N2-C1, 124.7(3).

FIG. 4 shows topographical steric maps of [Au(IPr#)Cl](9) and [Pd(IPr#)(cin)Cl](13) showing % V_{bur} per quadrant.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O(oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (242,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or C1, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. In certain embodiments, the hydrocarbyl is optionally substituted C₁₋₁₂ alkyl. In certain embodiments, the hydrocarbyl is optionally substituted C₂₋₁₂ alkenyl. In certain embodiments, the hydrocarbyl is optionally substituted C₂₋₁₂ alkynyl. In certain embodiments, the hydrocarbyl is optionally substituted C₃₋₁₂ cycloalkyl. In certain embodiments, the hydrocarbyl is optionally substituted C₁₋₁₂ heteroalkyl. In certain embodiments, the hydrocarbyl is optionally substituted C₁₋₁₂ alkoxy. In certain embodiments, the hydrocarbyl is optionally substituted C₆₋₁₄ aryl, and/or optionally substituted C₆₋₁₂ aryl, and/or optionally substituted C₆₋₁₀ aryl. In certain embodiments, the hydrocarbyl is optionally substituted C₂-C₁₂ heterocyclyl. In certain embodiments, the hydrocarbyl is optionally substituted C₄-C₁₂ heteroaryl. In certain embodiments, the hydrocarbyl is optionally substituted C₁₋₁₂ acyl.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

### Preparation of Compounds

Compounds of Formula I-III or otherwise described herein can be prepared by the general schemes described herein, using the synthetic method known by those skilled in the art. The following examples illustrate non-limiting embodiments of the compound(s) described herein and their preparation.

In various embodiments, a compound of Formula I, or a salt, solvate, geometric isomer, or stereoisomer thereof, is provided:

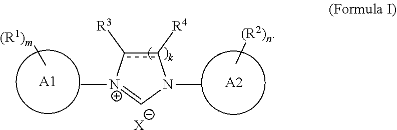

wherein:

[symbol] is a single or double bond;

A1 and A2 are each independently C₆₋₁₈ aryl or C₆₋₁₈ heteroaryl;

R¹ and R² are each independently C₁₋₃ alkyl substituted with at least one aryl or heteroaryl, wherein the aryl or heteroaryl in R¹ and R² is independently optionally substituted; in certain embodiments, the optional substituent is independently at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl;

R³ and R⁴ are each independently hydrogen, optionally substituted C₃₋₁₀ cycloalkyl, optionally substituted aryl, optionally substituted heteroaryl, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), or OC₁₋₁₂ alkyl; in certain embodiments, the optional substituent is independently at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl; or

R³ and R⁴ taken together with the ring to which they are attached are used to form a C₄₋₂₀ cycloalkyl, C₆₋₂₀ aryl, or C₆₋₂₀ heteroaryl, each of which is optionally substituted; in certain embodiments, the optional substituent is independently at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)S02N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl;

X is a counter anion;

R is independently at each occurrence hydrogen or C₁₋₁₀ hydrocarbyl (such as, but not limited to, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl);

k is 1, 2, 3, or 4;

m is an integer from 0 to 6;

n is an integer from 0 to 6; and

with the proviso that if A1 and A2 are phenyl, then at least one of m or n is 3, 4, or 5.

In some embodiments, the compound of Formula I, or a salt, solvate, geometric isomer, or stereoisomer thereof, is selected from the group consisting of:

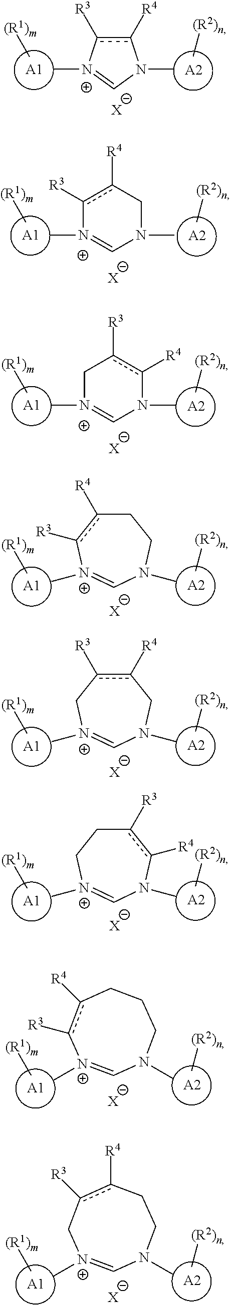

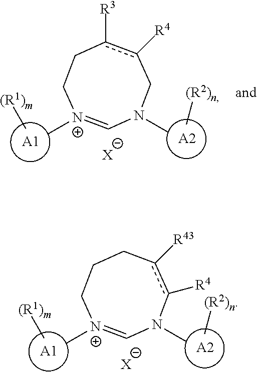

In various embodiments, A1 and A2 are identical. In various embodiments, A1 is a C₆₋₁₀ aryl. In some embodiments, A1 is phenyl. In other embodiments, A1 is naphthyl. In some embodiments, m is 3. In some embodiments, n is 3. In some embodiments, R¹ and R² are identical. In various embodiments, k is 1.

In some embodiments, R¹ is CH(aryl)₂. In certain embodiments, R¹ is CH(phenyl)₂. In certain embodiments, m is 3 and n is 3. In certain embodiments, X is an anionic counterion, bearing a −1 or a −2 charge. The type of X that can be used is not particularly limited, and any X that forms stable complexes with the compounds herein is suitable. In certain embodiments, X is selected from the group consisting of F, Cl, Br, I, OSO₂R, OSO₃R, OSO₂CF₃ (OTf), and OC(═O)R, and the like. In some embodiments, X is Cl.

In various embodiments, the compound of Formula I, or a salt, solvate, geometric isomer, or stereoisomer thereof, is selected from the group consisting of:

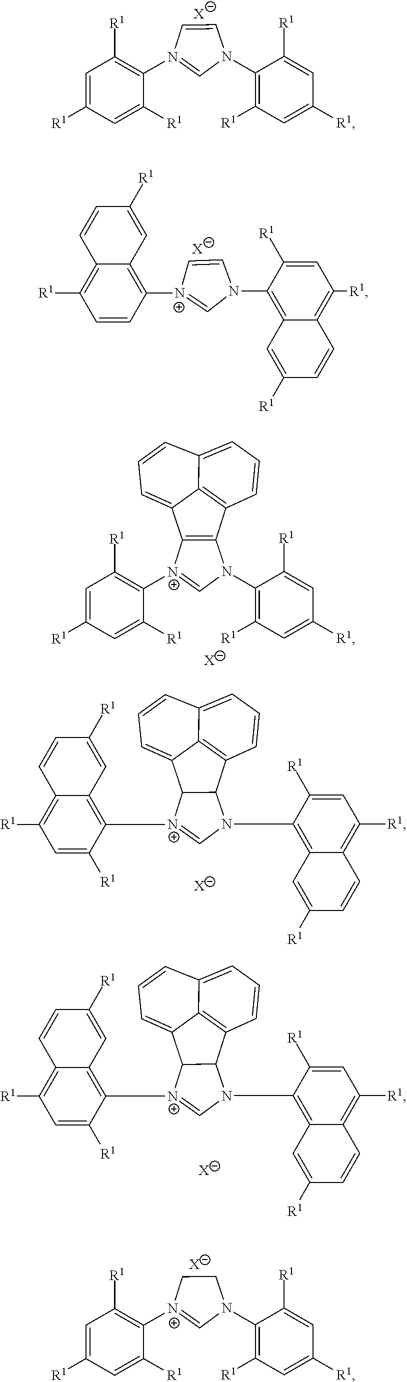

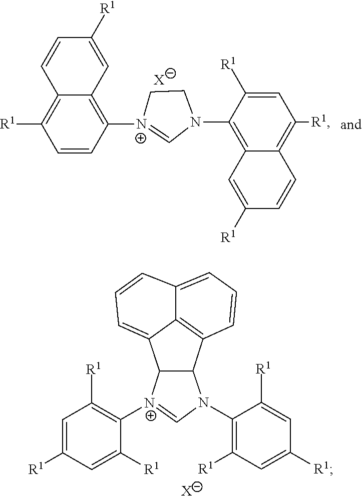

wherein R¹ is selected from the group consisting of CH(phenyl)₂, CH(4-Me-C₆H₄), CH(4-t-Bu-C₆H₄)₂, CH(4-MeO—C₆H₄)₂, CH(4-CF₃-C₆H₄)₂, CH(3,5-dimethyl-C₆H₃)₂, CH(3,5-ditrifluoromethyl-C₆H₃)₂, and CH(3,5-difluoro-C₆H₃)₂.

In one embodiment, a compound of Formula II, or a salt, solvate, geometric isomer, or stereoisomer thereof, is provided:

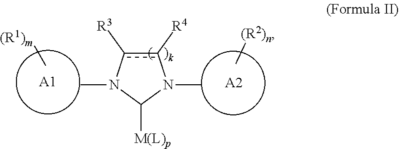

wherein:

M is an element of Group VIII to Group XVI with an atomic weight greater than 25;

L is a ligand of M, wherein at each occurrence L can be the same or different;

p is whole number from 0 to 5; and

[symbol] is a single or double bond;

A1 and A2 are each independently C₆₋₁₀ aryl or C₆₋₁₀ heteroaryl;

R¹ and R² are each independently C₁₋₃ alkyl substituted with at least one aryl or heteroaryl, wherein the aryl or heteroaryl in R¹ and R² is independently optionally substituted; in certain embodiments, the optional substituent is independently at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl;

R³ and R⁴ are each independently hydrogen, optionally substituted C₃₋₁₀ cycloalkyl, optionally substituted aryl, optionally substituted heteroaryl, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄₋₁₂ heteroaryl, or optionally substituted C₁₋₁₂ acyl), or OC₁₋₁₂ alkyl; in certain embodiments, the optional substituent is independently at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl; or

R³ and R⁴ taken together with the ring to which they are attached are used to form a C₄₋₂₀ cycloalkyl, C₆₋₂₀ aryl, or C₆₋₂₀ heteroaryl, each of which is optionally substituted; in certain embodiments, the optional substituent is independently at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)S02N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl;

R is independently at each occurrence hydrogen or C₁₋₁₀ hydrocarbyl (such as, but not limited to, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl);

k is 1, 2, 3, or 4;

m is an integer from 0 to 5;

n is an integer from 0 to 5; and

wherein, if A1 and A2 are phenyl, then at least one of m or n is 3, 4, or 5.

In various embodiments, M is selected from the group consisting of Fe, Co, Ni, Cu, Ru, Rh, Pd, Ag, Re, Os, Ir, Pt, and Au. In one embodiment, M is Pd. In one embodiment, M is a Group XVI element. In one embodiment, M is Se. In various embodiments, p is 0, 1, 2, 3, 4, or 5. When p is 0, ligand L is absent.

Ligand L represents one or more ligands whose number is determined by the valence of metal M and through variable ‘p’. When more than one ligand L is present, each L can be the same or different. For example, when p is 2, (L)₂ can be two identical ligands or two different ligands. In some embodiments, at least one L is an anionic ligand. In another embodiment, at least one L is a neutral ligand. The type of ligand L is not particularly limited, and any ligand L known in the art to form a complex or act as a counterion to M can be used. For example, and without limitation, anionic L's include halides, alkyl carboxylates, dicarboxylates, sulfates, sulfonates, CN, ferrocenes, and the like. In one embodiment at least one L is Cl. Neutral L's include, without limitation, CO, cyclooctadiene (COD), π-coordinated alkenes, π-coordinated aryl alkenes, and the like.

In various embodiments in the compound of Formula II, L is selected from the group consisting of acac, Cl, 3-Cl-pyridine, pyridine, N—R-imidazole (R is Me, Et, Bu, or Ph), cinnamyl, allyl, 1-R-indenyl (R=t-Bu, i-Pr, Et, Me, Cyclohexyl, 1-Adamantyl), Cp (cyclopentadienyl), aniline, 3-CF₃-aniline, μ-Cl, μ-OH, and 1,4-naphthoquinone; and p is 1, 2, or 3.

In various embodiments, the compound of Formula II is selected from the group consisting of [Pd(NHC)(acac)Cl], [Pd(NHC)(—Cl-pyridine)Cl₂], [Pd(NHC)(pyridine)Cl₂], [Pd(NHC)(N—R′-imidazole)Cl₂][Pd(NHC)(cinnamyl)Cl], [Pd(NHC)(allyl)Cl], [Pd(NHC)(1-R″-indenyl)Cl](R═), [Pd(NHC)(Cp)Cl](Cp=cyclopentadienyl), [Pd(NHC)(aniline)Cl₂], [Pd(NHC)(—CF₃-anlinine)Cl₂], [Pd(NHC)(μ-Cl)Cl]₂, [Pd(NHC)(μ-OH)Cl]₂, [Pd(NHC)(1,4-naphthoquinone)],

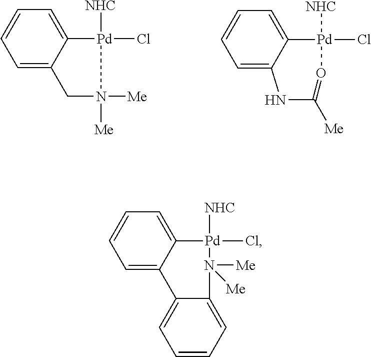

[Ni(NHC)CpCl], [Ni(NHC)(PR^{A}₃)X^{A}₂], [Ni(NHC)(di-tert-butyl-fumarate)₂], [Ni(NHC)(methyl methacrylate)₂], [Au(NHC)X^{B}], [Cu(NHC)X^{A}], [Ag(NHC)X^{A}],

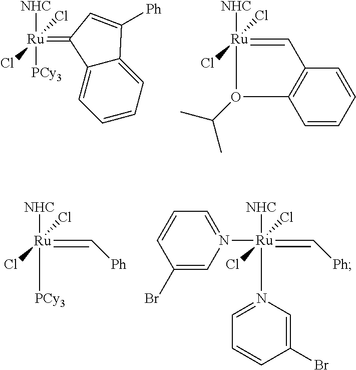

wherein NHC is selected from the group consisting of

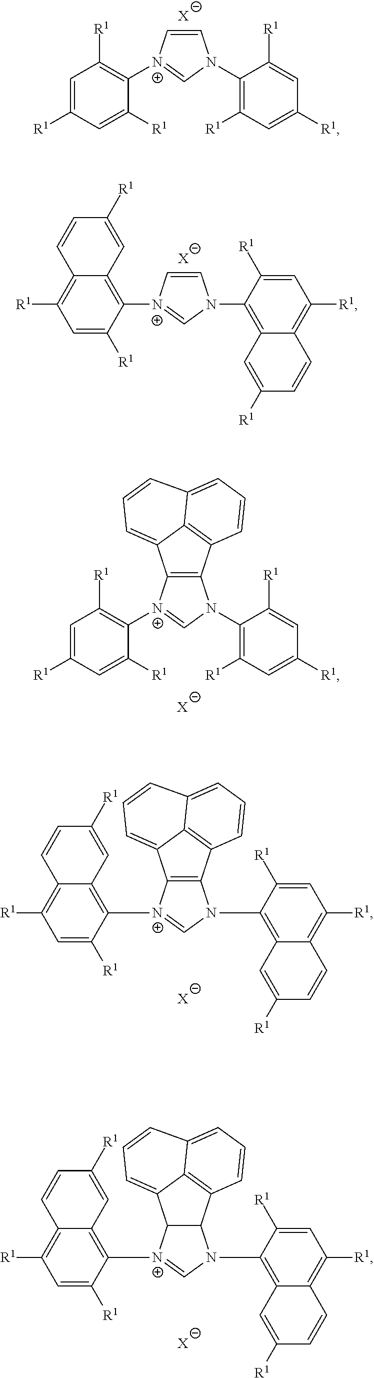

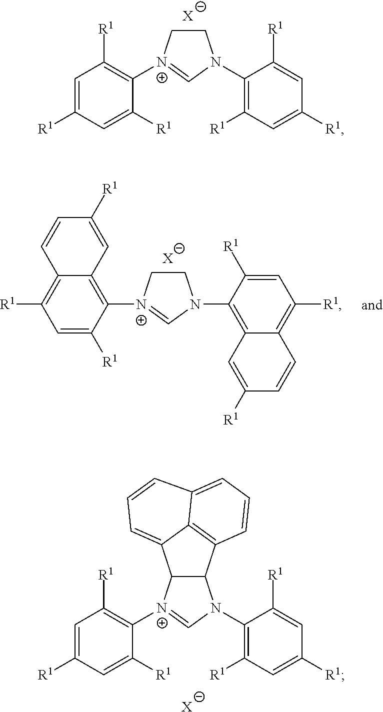

R′ is Me, Et, Bu, or Ph,

R″ is t-Bu, i-Pr, Et, Me, Cyclohexyl, or 1-Adamantyl;

X^{A} is Cl, Br, or I

X^{B} is Cl, Br, I, NTf₂, or OTf

PR^{A}₃, wherein each R^{A} is independently a C₁₋₈ hydrocarbyl (such as, but not limited to, optionally substituted C₁₋₈ alkyl, optionally substituted C₂₋₈ alkenyl, optionally substituted C₂₋₈ alkynyl, optionally substituted C₃₋₈ cycloalkyl, optionally substituted C₁₋₈ heteroalkyl, optionally substituted C₁₋₈ alkoxy, optionally substituted C₆ aryl, optionally substituted C₂-C₈ heterocyclyl, optionally substituted C₄-C₈ heteroaryl, or optionally substituted C₁₋₈ acyl) or a C₆₋₁₀ aryl;

R¹ is CH(phenyl)₂, CH(4-Me-C₆H₄), CH(4-t-Bu-C₆H₄)₂, CH(4-MeO—C₆H₄)₂, CH(4-CF₃-C₆H₄)₂, CH(3,5-dimethyl-C₆H₃)₂, CH(3,5-ditrifluoromethyl-C₆H₃)₂, or CH(3,5-difluoro-C₆H₃)₂.

In one embodiment, R^{A} is phenyl.

In one embodiment, a method of making the compound of Formula I, or a salt, solvate, geometric isomer, or stereoisomer thereof, is provided. The method includes contacting a compound with the structure

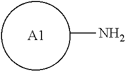

with a cationic form of R¹, to form a compound of Formula III:

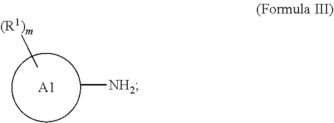

condensing the compound of Formula III with (CHO)₂ to form a diimine compound with the structure

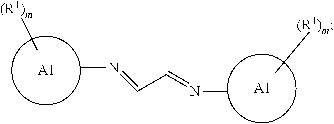

and cyclizing the diimine compound to form the compound of Formula I, or a salt, solvate, geometric isomer, or stereoisomer thereof.

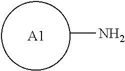

The compound of structure is an aryl or heteroaryl amine. The nature of A1-NH₂ is not particularly limited, provided that stable complexes with transition metals can be formed with the ligands described herein and the resulting transition metal complexes possess catalytic activity. Other suitable A1 moieties include anthracenes (e.g. 1-aminoanthracene, 2-aminoanthracene, 9-aminoanthracene); aminobiphenyls (e.g. 4-aminobiphenyl); aminophenanthrenes (e.g. 1-aminophenanthrene, 2-aminophenanthrene, 9-aminophenanthrene); aminopyrenes (e.g. 1-aminopyrene, 2-aminopyrene); aminochrysenes (e.g. 1-aminochrysene, 2-aminochrysene, 6-aminochrysene); aminofluorenes (e.g. 1-aminofluorene, 2-aminofluorene); naphthalenes (e.g. 1-aminonaphthalene, 2-aminonaphthalene); acridines (e.g. 9-aminoacridine, 2-aminoacridine); quinolines (e.g. 8-aminoquinoline, 2-aminoquinoline, 5-aminoquinoline); and the like.

A cationic form of R¹ includes solvated cations of R¹ and cation-anion complexes R¹ where R¹ can react as a cation. Cationic R¹ can be generated from suitable precursors, such as R¹—OH. Exposing R¹—OH to a protic acid and/or a Lewis Acid results in cationic R¹ that acts as an electrophile that reacts with A1, as defined herein. In one embodiment, the cationic form of R¹ is Ph₂CH⁺.

The method includes reacting the diimine compound with E-X, wherein E is an electrophile. When the diimine compound is reacted with E-X, the X becomes the counterion to the compound of Formula I as defined herein, and the electrophilic portion E reacts with solvent or other components of the reaction mixture and is ultimately discarded. Suitable electrophilic E groups include, without limitation, alkyl silanes.

In various embodiments, the compound is a compound of Formula IV, or a salt, solvate, geometric isomer, or stereoisomer thereof:

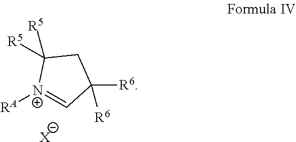

In the compound of Formula IV, X is as defined herein.

In certain embodiments, at least one occurrence of R^{A}, R⁵ or R⁶ is independently optionally substituted C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl). In certain embodiments, at least one occurrence of R^{A}, R⁵ or R⁶ is independently optionally substituted C₁₋₁₂ heteroalkyl. In certain embodiments, at least one occurrence of R^{A}, R⁵ or R⁶ is independently optionally substituted OC₁₋₁₂ alkyl. In certain embodiments, at least one occurrence of R^{A}, R⁵ or R⁶ is independently optionally substituted C₃₋₁₂ cycloalkyl. In certain embodiments, at least one occurrence of R^{A}, R⁵ or R⁶ is independently optionally substituted C₆₋₁₈ aryl. In certain embodiments, at least one occurrence of R^{A}, R⁵ or R⁶ is independently optionally substituted C₆₋₁₈ heteroaryl. In certain embodiments, at least one occurrence of R^{A}, R⁵ or R⁶ is independently A1. In certain embodiments, at least one occurrence of R^{A}, R⁵ or R⁶ is independently A1. In certain embodiments, at least one occurrence of R^{A}, R⁵ or R⁶ is independently R². In certain embodiments, the optional substitution in R⁵ and R⁶ is at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)S02N(R)₂, (CH₂)₀₋₂N(R)C(O)R, (CH₂)₀₋₂N(R)N(R)₂, N(R)C(O)OR, C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl), C₁₋₁₂ heteroalkyl, OC₁₋₁₂ alkyl, C₃₋₁₂ cycloalkyl, C₆₋₁₀ aryl, and C₆₋₁₀ heteroaryl.

Deprotonation of a compound of Formula IV results in the formation of a cyclic alkyl amino carbene (CAAC). Carbenes derived from the compound of Formula IV can form metal complexes of Formula IVa:

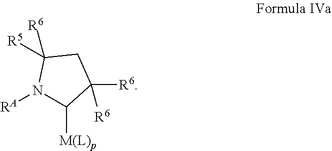

In the structure of Formula IVa, M, L, and ‘p’ are as defined herein.

In various embodiments, the compound is a compound of Formula V, or a salt, solvate, geometric isomer, or stereoisomer thereof:

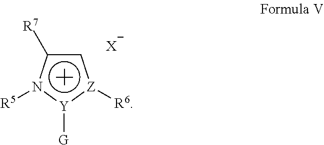

In the compound of Formula V, X, R⁵ and R⁶ are as defined herein. Variable R⁷ is defined the same as R⁵. Variable Y is N or C, Z is N or C, provided that both Y and Z cannot both be C. G is absent or defined the same as R⁵. Compounds of Formula V are mesoionic carbene precursors. Upon transmetallation, compounds of Formula V can form metal-carbene complexes of Formula Va:

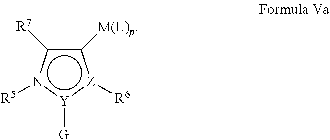

For example, a compound of Formula V can undergo the following reactions to form a compound of Formula Va:

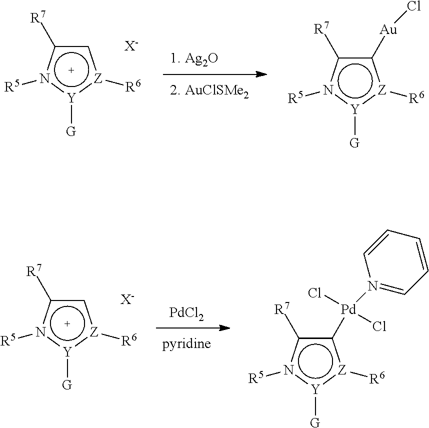

In the structure of Formula Va, M, L, and ‘p’ are as defined herein.

In various embodiments, the compound is a mesoionic carbene precursor is selected from the group consisting of:

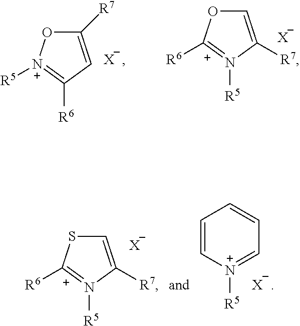

In certain embodiments, the optional substitution of any group contemplated herein comprises halogen. In certain embodiments, the optional substitution of any group contemplated herein comprises OR. In certain embodiments, the optional substitution of any group contemplated herein comprises SiR₃. In certain embodiments, the optional substitution of any group contemplated herein comprises OSiR₃. In certain embodiments, the optional substitution of any group contemplated herein comprises OSiR₃. In certain embodiments, the optional substitution of any group contemplated herein comprises OSi(OR)₃. In certain embodiments, the optional substitution of any group contemplated herein comprises BR₃. In certain embodiments, the optional substitution of any group contemplated herein comprises BR₂. In certain embodiments, the optional substitution of any group contemplated herein comprises B(OR)₃. In certain embodiments, the optional substitution of any group contemplated herein comprises B(OR)₂. In certain embodiments, the optional substitution of any group contemplated herein comprises CN. In certain embodiments, the optional substitution of any group contemplated herein comprises CF₃. In certain embodiments, the optional substitution of any group contemplated herein comprises OCF₃. In certain embodiments, the optional substitution of any group contemplated herein comprises SO₂R. In certain embodiments, the optional substitution of any group contemplated herein comprises SO₂N(R)₂. In certain embodiments, the optional substitution of any group contemplated herein comprises SO₃R. In certain embodiments, the optional substitution of any group contemplated herein comprises C(O)R. In certain embodiments, the optional substitution of any group contemplated herein comprises NR₂. In certain embodiments, the optional substitution of any group contemplated herein comprises N(R)SO₂R. In certain embodiments, the optional substitution of any group contemplated herein comprises N(R)SO₂N(R)₂. In certain embodiments, the optional substitution of any group contemplated herein comprises (CH₂)₀₋₂N(R)C(O)R. In certain embodiments, the optional substitution of any group contemplated herein comprises (CH₂)₀₋₂N(R)N(R)₂. In certain embodiments, the optional substitution of any group contemplated herein comprises N(R)C(O)OR. In certain embodiments, the optional substitution of any group contemplated herein comprises C₁₋₁₂ hydrocarbyl (such as, but not limited to, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl). In certain embodiments, the optional substitution of any group contemplated herein comprises C₁₋₁₂ heteroalkyl. In certain embodiments, the optional substitution of any group contemplated herein comprises OC₁₋₁₂ alkyl. In certain embodiments, the optional substitution of any group contemplated herein comprises C₃₋₁₂ cycloalkyl. In certain embodiments, the optional substitution of any group contemplated herein comprises C₆₋₁₀ aryl. In certain embodiments, the optional substitution of any group contemplated herein comprises C₆₋₁₀ heteroaryl.

The compounds described herein can possess one or more stereocenters, and each stereocenter can exist independently in either the (R) or (5) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

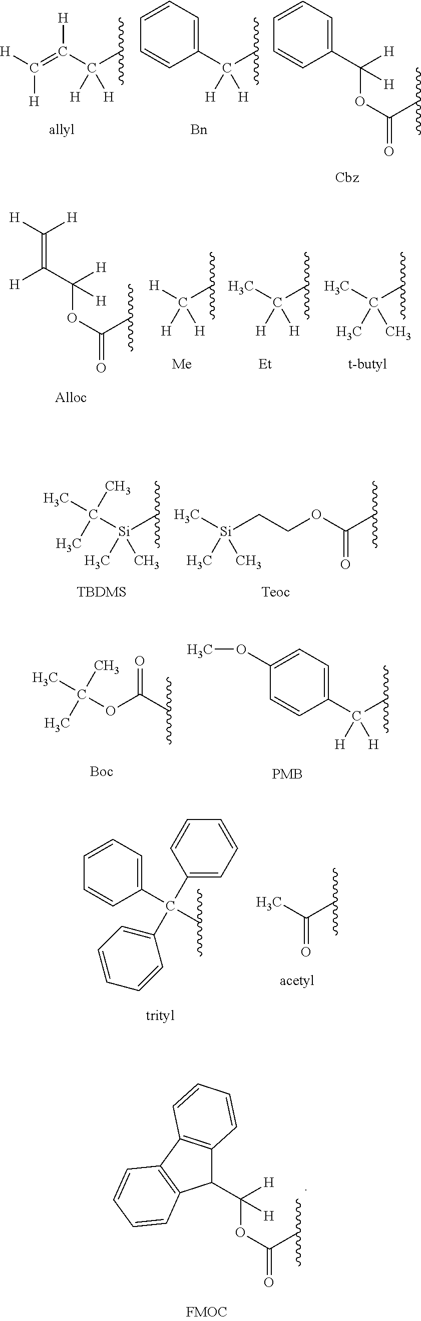

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and

Kocienski, Protective Groups, Thieme Verlag, New York, N.Y., 1994, which are incorporated herein by reference for such disclosure.

### Examples

Various embodiments of the present application can be better understood by reference to the following Examples which are offered by way of illustration. The scope of the present application is not limited to the Examples given herein.

The synthesis of compounds of Formula I, such as compound 5, also designated as “IPr#” herein, is shown in Scheme 1. It should be noted that the direct three-fold alkylation of aniline is significantly more challenging than the alkylation of para-blocked toluidine as a consequence of N—/C-alkyl migration. After experimentation, it was unexpectedly discovered that the previously unknown 2,4,6-tribenzhydrylaniline 7 could be prepared in 79% yield (93 g, 200 mmol scale) by adding HCl (1.0 equiv) to a solution of aniline (1.0 equiv), diphenylmethanol (3.5 equiv) and ZnCl2 (0.5 equiv) at 160° C. Routinely, 70-75% yields were obtained on 10-20 mmol scale. This represents a significant improvement over the previous Friedel-Crafts method involving addition of a mixture of ZnCl2 and HCl at 160° C., which led to irreproducible results. Without being bound by theory, it is believed that alkylation of aniline at para position occurs last; the N-alkylation products were not observed. The synthesis of diimine 8 was smoothly effected by reacting glyoxal (1.0 equiv) with amine 7 (2.0 equiv) and MgSO₄ (2.5 equiv) (70 g, 160 mmol scale). The reaction was slower than in the synthesis of IPr*, suggesting more pronounced steric character. The diimine was formed as exclusively the s-trans isomer. The cyclization step occurred smoothly upon exposing the diimine (1.0 equiv) and paraformaldehyde (1.1 equiv) to TMSC1 (1.1 equiv) in EtOAc at 70° C. (25 g, 40 mmol scale). In some embodiments, this procedure allows for much milder cyclization to 5 than the HCl/ZnCl2 combination, which again proved problematic for large-scale runs and was riddled with retro-Friedel-Crafts products. The TMSC1 procedure could be conveniently followed by color change from yellow to light grey, indicating completion of the reaction.

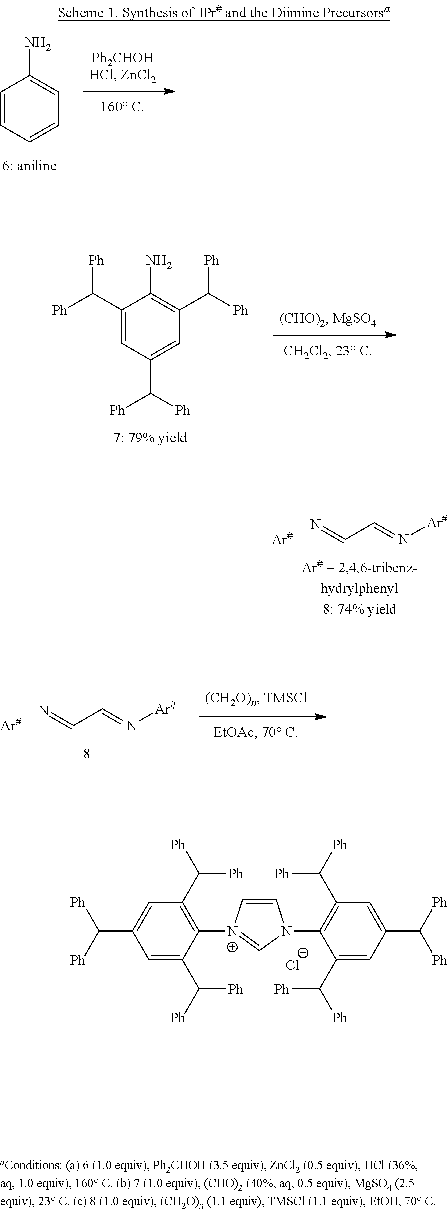

In various embodiments, (1) the three steps do not require purification of the intermediates, (2) the final product is obtained after facile work-up (filtration), (3) the procedure uses industrial chemicals available in bulk, (4) the sequence is routinely performed within two days.

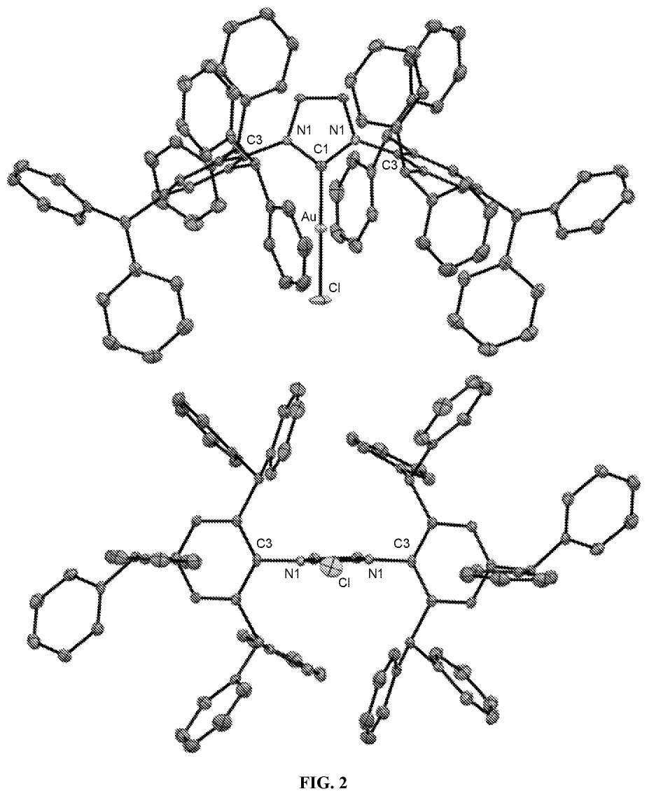

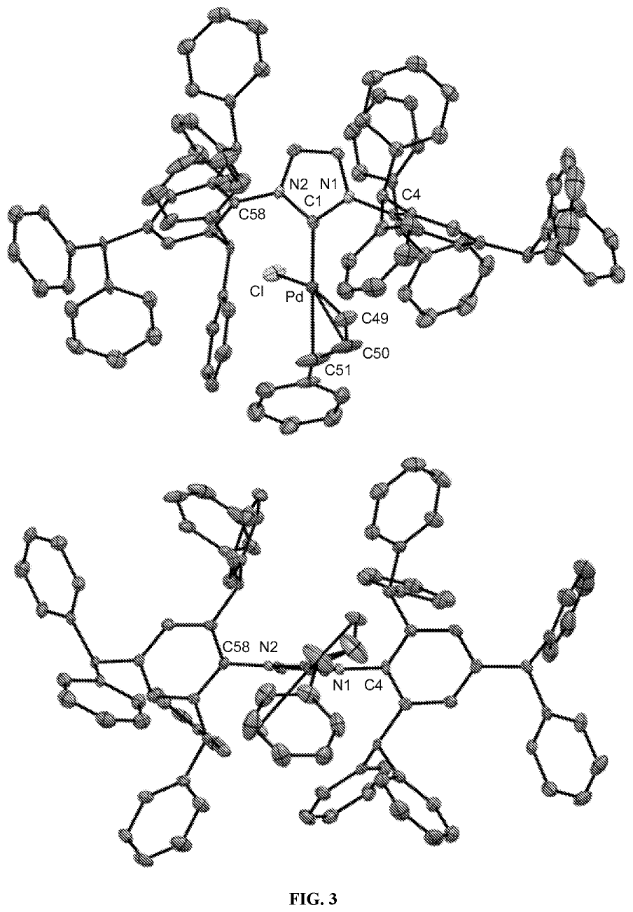

With multigram access to IPr# secured, we next comprehensively evaluated steric and electronic properties of this novel NHC ligand. As shown in Scheme 2, the gold complex [Au(IPr#)Cl](9) was prepared using the general method disclosed by Nolan, while Rh(I) complexes, [Rh(IPr#)(CO)₂Cl](10) and [Rh(IPr#)(acac)CO] (11) were prepared after generating the free carbene in situ by deprotonation of IPr#HCl with a slight excess of either KHMDS or KOt-Bu. The Rh(I) complex (10) could be prepared either directly by using rhodium dicarbonyl chloro dimer [{Rh(CO)₂(μ-Cl)}2] (path b) or in a mild two-step procedure via [Rh(IPr#)(cod)Cl] and the reaction with carbon monoxide (path c). In some embodiments, the selenium adduct can be generated [Se(IPr#)] (12) by adding the free carbene generated in situ to excess of selenium. The Pd(II) complex [Pd(IPr#)(cin)Cl] (13) was prepared by generating the free carbene in situ and coordinating to the palladium cinnamyl dimer [{Pd(cin)(μ-Cl)}2]. In various embodiments, all complexes are stable to air and moisture. Complexes 9 and 13 were fully characterized by X-ray crystallography (FIGS. 2 and 3).

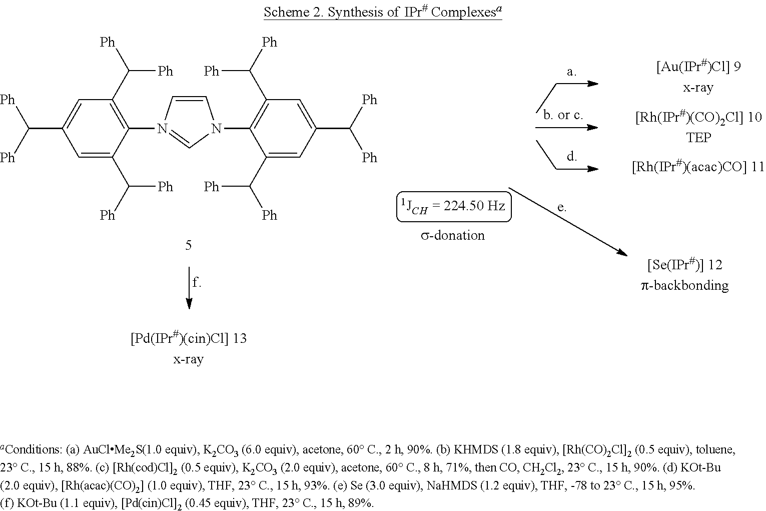

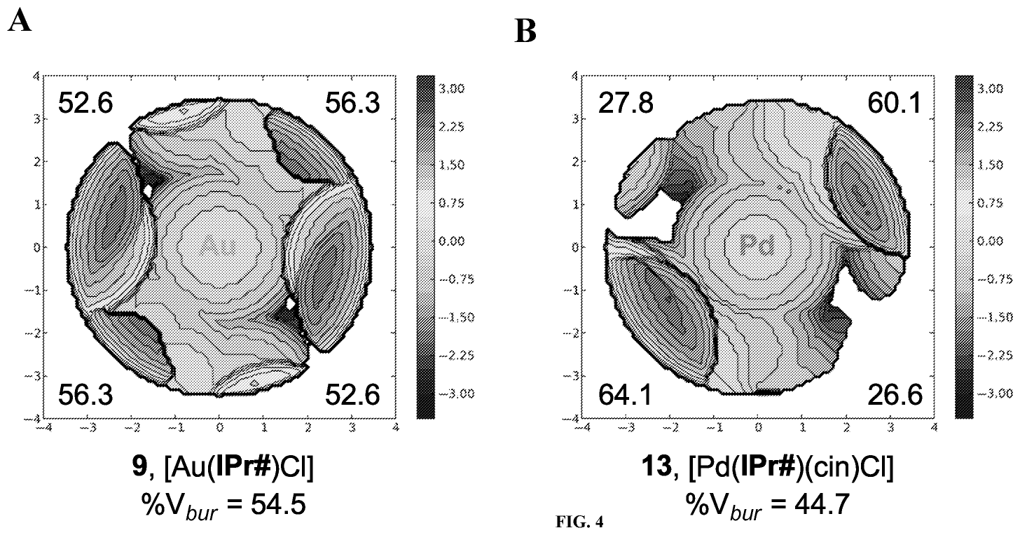

Some prior studies demonstrated the % buried volume (% V_{bur}) and steric maps of model [Au(NHC)Cl] complexes as the best indication of quantifying the steric impact of NHC ligands. In one embodiment, [Au(IPr#)Cl] is linear (C—Au—Cl, 180.0°; C—Au, 1.972 Å), making it a good model for evaluating % V_{bur}. Thus, with the (% V_{bur}) of 54.5%, [Au(IPr#)Cl] represents one of the bulkiest NHC ligands prepared to date (Table 1). This value compares well with the (% V_{bur}) of 50.4% determined for [Au(IPr*)Cl] (C—Au—Cl, 178.3°; C—Au, 1.987 Å), indicating a subtle but important effect of the para-diphenylmethyl substitution on the steric properties of the ligand. A graphical representation of the steric mapping of the metal center in [Au(IPr#)Cl] is shown in FIG. 4A.

TABLE 1
Summary of Steric and Electronic Parameters

| | % V_{bur} | TEP | δ(⁷⁷Se) | ¹J_{CH} |
|---|---|---|---|---|
| NHC | [Au] | [cm⁻¹] | [ppm] | [Hz] |
| IPr# | 54.5 | 2051.8 | 108 | 224.50 |
| IPr | 45.4 | 2051.5 | 90 | 223.70 |
| IPr* | 50.4 | 2052.7 | 106 | 224.99 |
| CAAC^{Cy} | 51.0^{a} | 2048.6 | 492^{b} | 188.53 |

^{a}Menthyl instead of cyclohexyl.
^{b}Me₂ instead of cyclohexyl.

The Tolman electronic parameter (TEP) allows to evaluate electronic properties of NHC ligands. Thus, the CO stretching frequencies of [Rh(IPr#)(CO)₂Cl] are v_{sym}=2079.5 cm⁻¹ and v_{asym}=1999.5 cm⁻¹ (CH₂Cl₂, 0.20 M), respectively, which corresponds to a TEP of 2051.8 cm⁻¹ as a combined measure of the electronic properties of the ligand. These values match well with the IPr ligand (TEP of 2051.5 cm⁻¹), IPr* (TEP of 2052.7 cm⁻¹) and CAA^{Cy} (TEP of 2048.6 cm⁻¹) and indicate one of the most donating 5-membered NHCs prepared.

Likewise, the use of selenourea adducts permits to evaluate π-backbonding from the ⁷⁷Se NMR spectra. The δₛₑ value of 108.11 ppm for [Se(IPr#)] (CDCl₃) suggests that the expanded substitution leads to slightly higher π-accepting properties than IPr (δₛₑ=90 ppm), and IPr* (δₛₑ=106 ppm) and much lower than CAAC^{Me2} (δₛₑ=492 ppm), as expected from the C-substitution.

Furthermore, one-bond CH J coupling constants obtained from ¹³C satellites of the ¹H NMR spectrum provide good indication of σ-donating properties of an NHC ligand. The value of 224.50 Hz for IPr#HCl (CDCl₃) is consistent with this ligand being as strongly 6-donating as IPr(¹J_{CH}=223.70 Hz), but at the same time significantly more sterically-demanding and flexible. The chemical shift of the iminium proton in IPr#HCl was found at 12.6 ppm (CDCl₃), which is significantly downfield compared with other imidazolium salts.

The synthesis of [Rh(IPr#)(acac)CO] demonstrates that the extremely bulky IPr# is able to accommodate asymmetrical, κ²—O,O-bound ligands like acac to the metal center. In certain embodiments, this leads to steric adjustment of the ligand topology to fit the Rh coordination plane.

This property is confirmed through the synthesis and full crystallographic characterization of [Pd(IPr#)(cin)Cl] (FIG. 3). Following the elegant reports from Nolan and Hazari, Pd(II) allyl-type complex (13) was selected as a model well-defined, air- and moisture-stable Pd(II)—NHC precatalyst to evaluate the performance of IPr# in cross-coupling reactions. The X-ray crystallographic analysis revealed the (% V_{bur}) of 44.7% with 64.1%, 27.8%, 60.1%, 26.6% for each quadrant (FIG. 4B). The values can be compared with the (% V_{bur}) of 54.5% for the linear [Au(IPr#)Cl] with 56.3%, 52.6%, 56.3%, 52.6% for each quadrant (FIG. 4A). Thus, the IPr# ligand is capable of both (1) adjusting the steric environment, and (2) asymmetrical twisting around the metal center, which furnishes important in catalysis differentiated quadrant distribution arising from the very bulky yet flexible ligand topology. The C—Pd, Pd—Cl, and Pd—C(Ph) bond lengths of 2.044 Å, 2.374 Å, and 2.210 Å in 13 are in the range for Pd(II)-allyl type complexes ([Pd(IPr)(cin)Cl], C—Pd, 2.027 Å; Pd—Cl, 2.357 Å; Pd—C(Ph), 2.245 Å).

It is further worth noting that diphenylmethyl substituents of the IPr# wingtips extend beyond the metal center in both 9 and 13, which might influence both (1) substrate approach, and (2) coordination of active intermediates formed during the catalysis.

The activity of IPr# was evaluated in various palladium-catalyzed cross-couplings (Scheme 3). As stated above, [Pd(IPr#)(cin)Cl] was selected due to the success of well-defined Pd(II)—NHCs supported by allyl-type throw-away ligands and the potential to tune the catalyst activity by allyl modifications. The results outlined in Scheme 3 indicate very high degree of generality of IPr#. As such, amide N—C(O) Suzuki cross-coupling (entry 1), ester C—O amidation (entry 2), amide N—C(O) transamidation (entry 3), C—Cl Suzuki cross-coupling (entry 4), C—Cl Buchwald-Hartwig amination (entry 5), C—Br Feringa coupling using both aryl-(entry 6) and challenging alkyllithium possessing β-hydrogens (entry 7), C—Cl ketone α-arylation (entry 8), C—S sulfur metathesis (entry 9) and C—H activation (entry 10) all proceeded in good to excellent yields. More importantly, these results demonstrate that the IPr# ligand is effective in an array of N—C, O—C, C—Cl, C—Br, C—S and C—H bond cross-couplings with various organometallics (B, Li, enolate, amine, sulfide) across some of the most broadly employed cross-couplings in industrial and academic settings.

Generality and two further applications of the IPr# ligand design concept are presented in Scheme 4. In various embodiments, compounds of Formula I such as BIAN-IPr# (14) and Np# (15) showcase the synthetic potential of the “hash” modular framework described herein. Thus, BIAN (BIAN=bis(imino)acenaphthalene) ligands have emerged as powerful ligands in catalysis because of the structural rigidity of C—H bonds bringing the wingtip substituents closer to the metal center as well as redox-active properties, and more pronounced σ-donating character of the carbene center. With the access to 2,4,6-tribenzhydrylaniline 7 in hand, the synthesis of BIAN-IPr# (14) proceeded uneventfully readily furnishing 1.5 gram of 14 using acenaphthoquinone (16) as the NHC precursor.

In the similar vein, the C₂-symmetric imidazolin-2-ylidenes bearing substituted naphthyl chains reported by Dorta have emerged as the most active NHC ancillary ligands in Pd-catalyzed cross-coupling and Ru-metathesis; however, the synthesis of 2,7-substituted naphthyl wingtips has been a limitation.

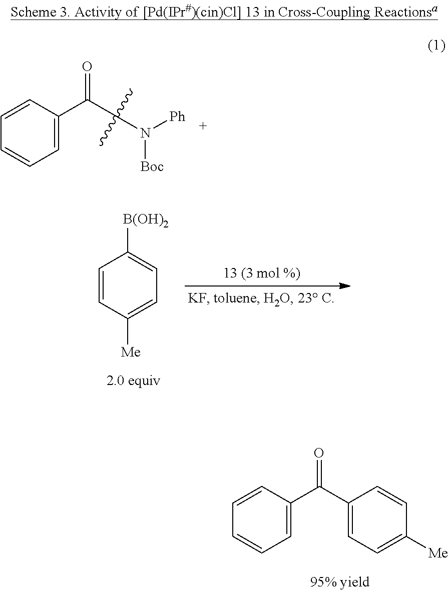

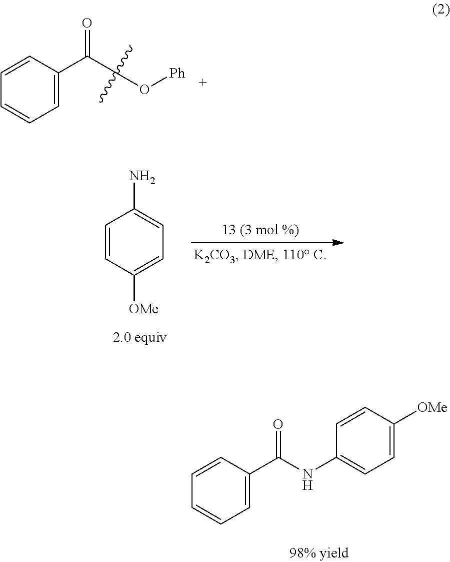

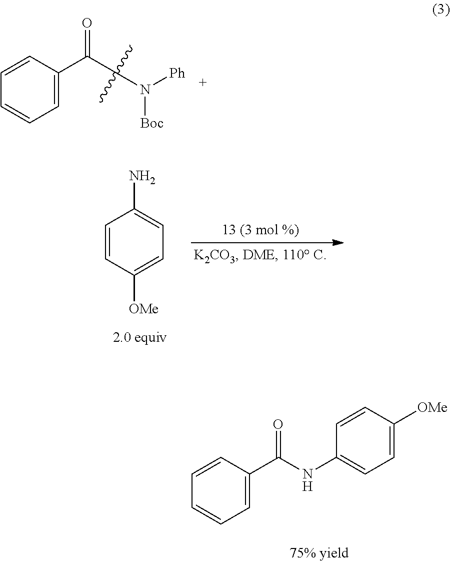

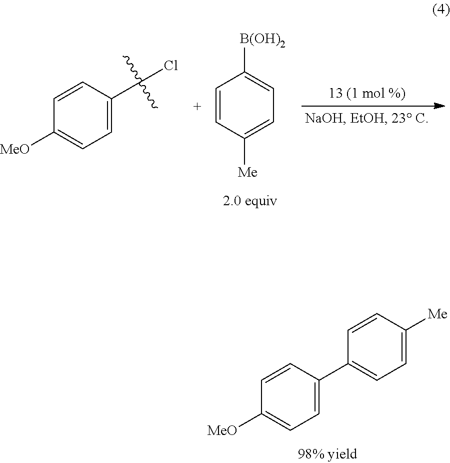

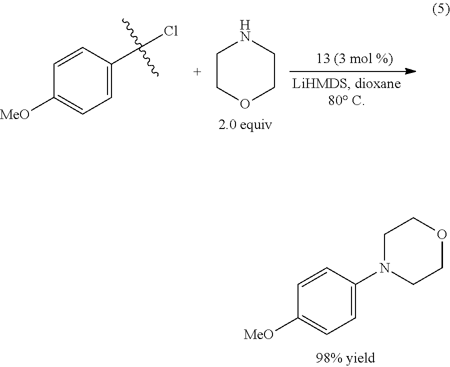

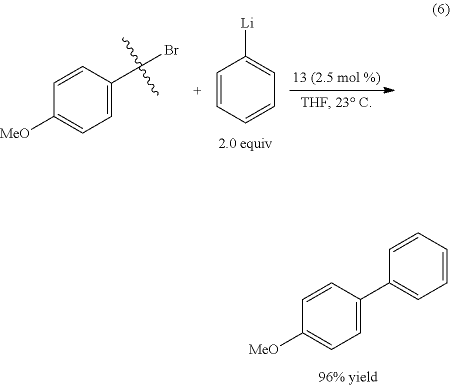

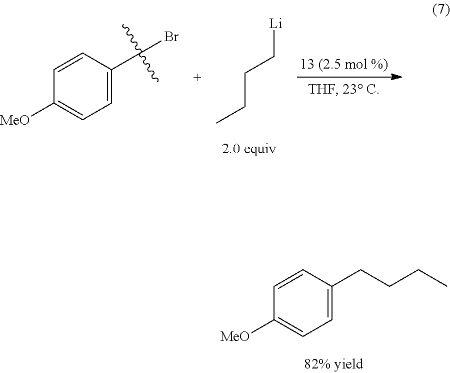

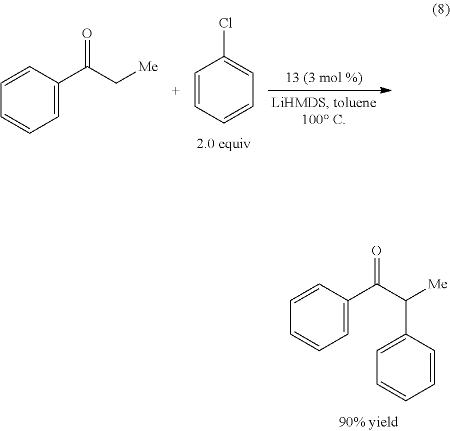 [SMILES: CCC(=O)c1ccccc1 ; Clc1ccccc1 ; CC(C(=O)c1ccccc1)c1ccccc1]

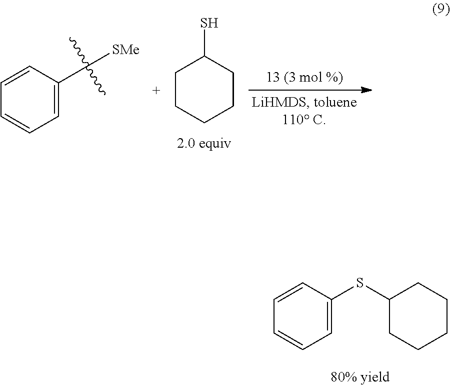

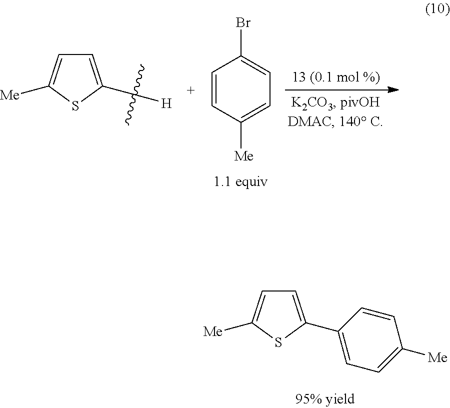

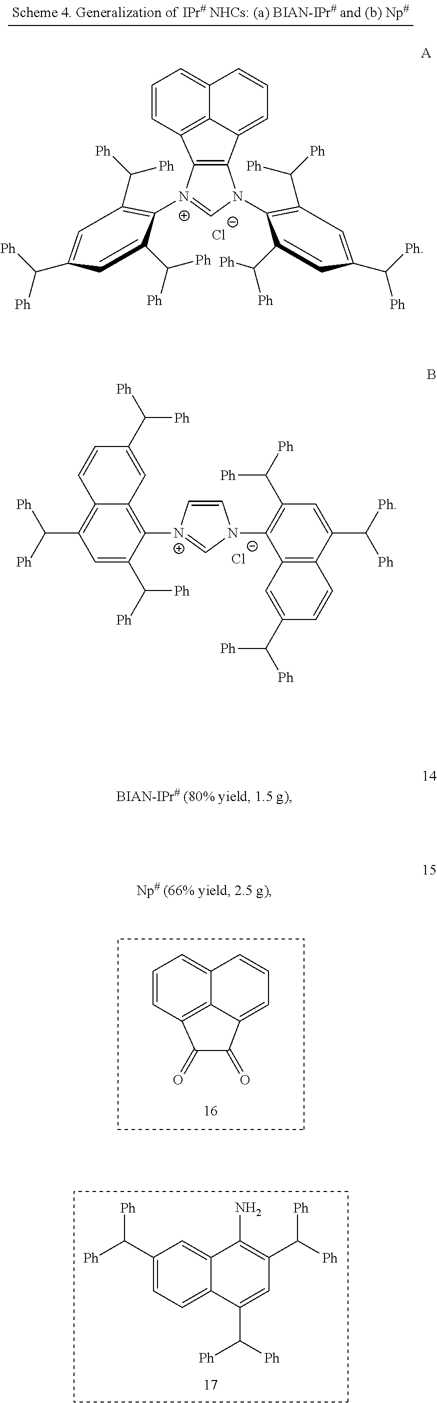

TABLE 2
HOMO and LUMO Energy Levels (eV) of IPr^{#} Calculated
at the B3LYP 6-311++g(d,p) Level

| NHC | HOMO [eV] | LUMO [eV] |
|---|---|---|
| IPr# | −6.16 | −0.96 |
| Np# | −6.04^{a} | −1.55^{a} |
| IPr* | −6.12 | −0.90 |
| IPr | −6.01 | −0.53 |
| IMes | −5.90 | −0.48 |

^{a}rac-Np# (C2-symmetric).
meso-Np# (C_{S}-symmetric), −5.97 eV and −1.57 eV.
rac-Np# is more stable than meso-Np# by 0.56 kcal/mol calculated at the B3LYP 6-311++g(d,p) level

Applying the concept described herein, the synthesis of Np# (15) exploiting the facile synthesis of 2,4,7-tribenzhydrylnaphthalen-1-amine (17) by Friedel-Crafts alkylation proceeded uneventfully and furnished 2.5 g of this sterically-differentiated NHC ligand. Thus, the use of “hash” concept permits a modular, rapid and cost-effective ligand assembly that might be applicable to both (1) various carbene classes, and (2) diverse amines (cf. Np#).

To further assess the effect of substitution on electronic properties of 5, HOMO and LUMO energy levels of IPr# and classical NHCs were determined at the B3LYP 6-311++g(d,p) level of theory (Table 2). Computation of HOMO and LUMO provides the most accurate estimation of nucleophilicity (more σ-donating, higher HOMO) and electrophilicity (more π-accepting, lower LUMO) of NHCs, however, the values for comparison must be available at the same level of theory.

The HOMO of IPr# (−6.16 eV) is comparable with IPr (−6.01 eV), which is a routine model for σ-donating NHCs. Replacement of the N-phenyl ring with N-naphthyl renders nucleophilicity of Np# similar to classic NHCs (HOMO, -6.04 eV), but with enhanced electrophilicity (LUMO, -1.55 eV). Thus, it is evident that in combination with differentiated steric impact, the class of IPr# ligands is well-suited for electronic fine-tuning of their properties for homogeneous catalysis.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a compound of Formula I, or a salt, solvate, geometric isomer, or stereoisomer thereof:

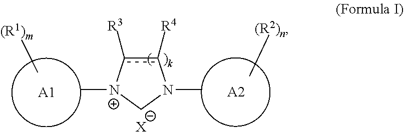

wherein: [symbol] is a single or double bond; A1 and A2 are each independently C₆₋₁₈ aryl or C₆₋₁₈ heteroaryl; R¹ and R² are each independently C₁₋₃ alkyl substituted with at least one aryl or heteroaryl, wherein the aryl or heteroaryl in R¹ and R² is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; R³ and R⁴ are each independently hydrogen, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl, wherein the optional substitution independently comprises at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; or R³ and R⁴ taken together with the ring to which they are attached are used to form a C₄₋₂₀ cycloalkyl, C₆₋₂₀ aryl, or C₆₋₂₀ heteroaryl, each of which is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; X is a counter anion; R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃-10 cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl; k is 1, 2, 3, or 4; m is an integer from 0 to 6; n is an integer from 0 to 6; and with the proviso that if A1 and A2 are phenyl, then at least one of m or n is 3, 4, or 5.

Embodiment 2 provides the compound of Embodiment 1, wherein A1 and A2 are identical.

Embodiment 3 provides the compound of any of Embodiments 1-2, wherein A1 is a C₆₋₁₀ aryl.

Embodiment 4 provides the compound of any of Embodiments 1-3, wherein A1 is phenyl.

Embodiment 5 provides the compound of any of Embodiments 1-3, wherein A1 is naphthyl.

Embodiment 6 provides the compound of any of Embodiments 1-5, wherein at least one of the following applies: m is 3, n is 3, or k is 1.

Embodiment 7 provides the compound of any of Embodiments 1-6, wherein the compound is selected from the group consisting of

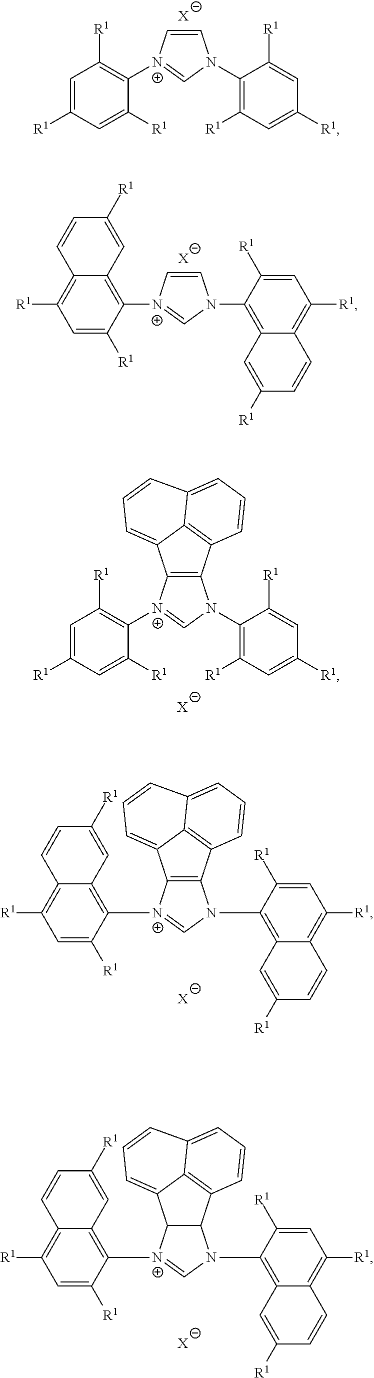

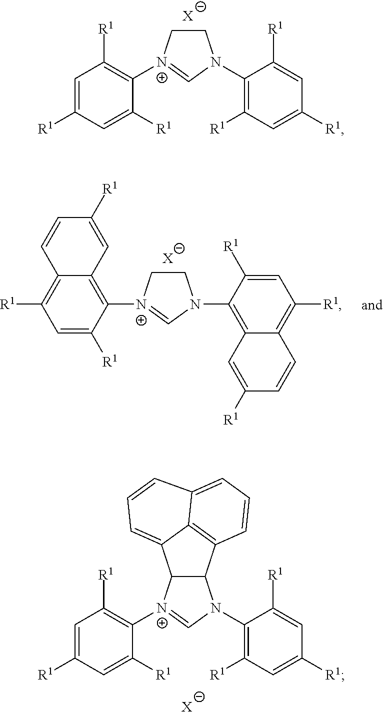

wherein R¹ is selected from the group consisting of CH(phenyl)₂, CH(4-Me-C₆H₄), CH(4-t-Bu-C₆H₄)₂, CH(4-MeO—C₆H₄)₂, CH(4-CF₃-C₆H₄)₂, CH(3,5-dimethyl-C₆H₃)₂, CH(3,5-ditrifluoromethyl-C₆H₃)₂, and CH(3,5-difluoro-C₆H₃)₂.

Embodiment 8 provides the compound of any of Embodiments 1-7, wherein X is C₁.

Embodiment 9 provides the compound of any of Embodiments 1-8, wherein R¹ and R² are identical.

Embodiment 10 provides the compound of any of Embodiments 1-9, wherein R¹ is CH(aryl)₂.

Embodiment 11 provides the compound of any of Embodiments 1-10, wherein R¹ is CH(phenyl)₂.

Embodiment 12 provides the compound of any of Embodiments 1-11, wherein m is 3 and n is 3.

Embodiment 13 provides the compound of any of Embodiments 1-12, wherein X is selected from the group consisting of F, Cl, Br, I, OSO₂R, OSO₃R, and OC(═O)R.

Embodiment 14 provides the compound of any of Embodiments 1-13, wherein X is C₁.

Embodiment 15 provides a compound of Formula II, or a salt, solvate, geometric isomer, or stereoisomer thereof:

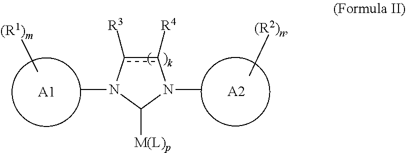

wherein: M is an element of Group VIII to Group XVI with an atomic weight greater than 25; L is a ligand of M, wherein at each occurrence L can be the same or different; p is whole number from 0 to 5; and [symbol] is a single or double bond; A1 and A2 are each independently C₆₋₁₀ aryl or C₆₋₁₀ heteroaryl; R¹ and R² are each independently C₁₋₃ alkyl substituted with at least one aryl or heteroaryl, wherein the aryl or heteroaryl in R¹ and R² is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; R³ and R⁴ are each independently hydrogen, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl, wherein the optional substitution independently comprises at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; or R³ and R⁴ taken together with the ring to which they are attached are used to form a C₄₋₂₀ cycloalkyl, C₆-20 aryl, or C₆₋₂₀ heteroaryl, each of which is independently optionally substituted by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃-10 cycloalkyl, optionally substituted C₁₋₁₀ heteroalkyl, optionally substituted C₁-10 alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl; k is 1, 2, 3, or 4; m is an integer from 0 to 5; n is an integer from 0 to 5; and with the proviso that if A1 and A2 are phenyl, then at least one of m or n is 3, 4, or 5.

Embodiment 16 provides the compound of Embodiment 15, wherein M is selected from the group consisting of Fe, Co, Ni, Cu, Ru, Rh, Pd, Ag, Re, Os, Ir, Pt, and Au.

Embodiment 17 provides the compound of any of Embodiments 15-16, wherein M is a Group XVI element.

Embodiment 18 provides the compound of any of Embodiments 15-17, wherein p is 0, 1, or 2.

Embodiment 19 provides the compound of any of Embodiments 15-18, wherein at least one L is an anionic ligand.

Embodiment 20 provides the compound of any of Embodiments 15-19, wherein at least one L is a neutral ligand.

Embodiment 21 provides the compound of any of Embodiments 15-20, wherein at least one L is selected from the group consisting of acac, Cl, 3-Cl-pyridine, pyridine, N—R′-imidazole, cinnamyl, allyl, 1-R″-indenyl, Cp (cyclopentadienyl), aniline, 3-CF₃-aniline, μ-Cl, μ-OH, 1,4-naphthoquinone, CpCl, PR^{A}₃, di-tert-butyl-fumarate, and methyl methacrylate; wherein R′ is Me, Et, Bu, or Ph; R″ is t-Bu, i-Pr, Et, Me, Cyclohexyl, or 1-Adamantyl; and PR^{A}₃, wherein each R^{A} is independently optionally substituted C₁₋₈ alkyl, optionally substituted C₂₋₈ alkenyl, optionally substituted C₂₋₈ alkynyl, optionally substituted C₃₋₈ cycloalkyl, optionally substituted C₁₋₈ heteroalkyl, optionally substituted C₁₋₈ alkoxy, optionally substituted C₆₋₁₀ aryl, optionally substituted C₂-C₁₀ heterocyclyl, optionally substituted C₄-C₁₀ heteroaryl, or optionally substituted C₁₋₁₂ acyl.

Embodiment 22 provides the compound of any of Embodiments 15-21, wherein the compound is selected from the group consisting of [Pd(NHC)(acac)Cl], [Pd(NHC)(3-Cl-pyridine)Cl₂], [Pd(NHC)(pyridine)Cl₂], [Pd(NHC)(N—R′-imidazole)Cl₂][Pd(NHC)(cinnamyl)Cl], [Pd(NHC)(allyl)Cl], [Pd(NHC)(1-R″-indenyl)Cl] (R═), [Pd(NHC)(Cp)Cl] (Cp=cyclopentadienyl), [Pd(NHC)(aniline)Cl₂]), [Pd(NHC)(3-CF₃-anlinine)Cl₂], [Pd(NHC)(μ-Cl)Cl]₂, [Pd(NHC)(μ-OH)Cl]₂, [Pd(NHC)(1,4-naphthoquinone)],

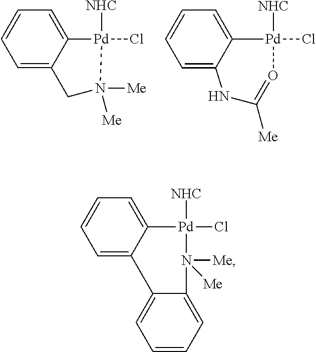

[Ni(NHC)CpCl], [Ni(NHC)(PR^{A}3)X^{A}2], [Ni(NHC)(di-tert-butyl-fumarate)₂], [Ni(NHC)(methyl methacrylate)_{2],} [Au(NHC)X¹³], [Cu(NHC)X^{A}], [Ag(NHC)X^{A}],

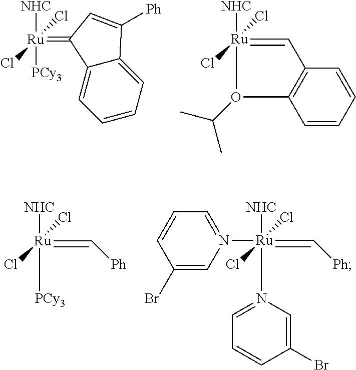

wherein NHC is selected from the group consisting of

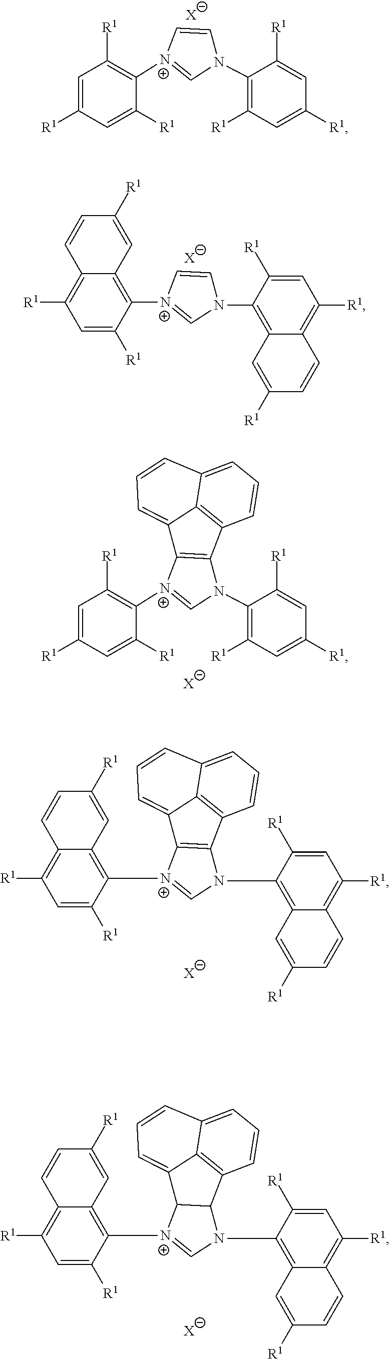

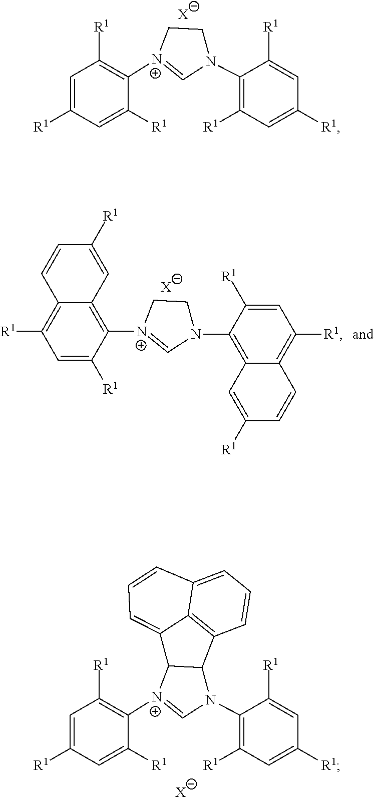

R¹ is selected from the group consisting of CH(phenyl)₂, CH(4-Me-C₆H₄), CH(4-t-Bu-C₆H₄)₂, CH(4-MeO—C₆H₄)₂, CH(4-CF₃-C₆H₄)₂, CH(3,5-dimethyl-C₆H₃)₂, CH(3,5-ditrifluoromethyl-C₆H₃)₂, and CH(3,5-difluoro-C₆H₃)₂;

R′ is Me, Et, Bu, or Ph;

R″ is t-Bu, i-Pr, Et, Me, Cyclohexyl, or 1-Adamantyl;

PR^{A}₃, wherein each R^{A} is independently optionally substituted C₁₋₈ alkyl, optionally substituted C₂₋₈ alkenyl, optionally substituted C₂₋₈ alkynyl, optionally substituted C₃₋₈ cycloalkyl, optionally substituted C₁₋₈ heteroalkyl, optionally substituted C₁₋₈ alkoxy, optionally substituted C₆₋₁₀ aryl, optionally substituted C₂-C₈ heterocyclyl, optionally substituted C₄-C₈ heteroaryl, or optionally substituted C₁₋₁₂ acyl;

X is Cl or Br;

X^{A} is Cl, Br, or I; and

X^{B} is Cl, Br, I, NTf₂, or OTf.

Embodiment 23 provides a method of making the compound of any of Embodiments the method comprising: contacting a compound with the structure

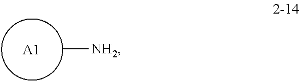

with a cationic form of R¹, to form a compound of Formula III, or a salt, solvate, geometric isomer, or stereoisomer thereof:

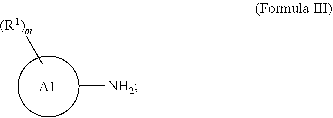

condensing the compound of Formula III with (CHO)₂ to form a diimine compound with the structure

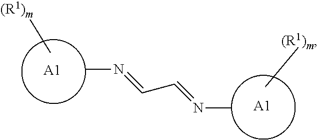

or a salt, solvate, geometric isomer, or stereoisomer thereof; and cyclizing the diimine compound to form the compound of Formula I, or a salt, solvate, geometric isomer, or stereoisomer thereof.

Embodiment 24 provides the method of Embodiment 23, wherein the cationic form of R¹ is Ph₂CH⁺.

Embodiment 25 provides the method of any of Embodiments 23-24, wherein the cyclizing comprises reacting the diimine compound with E-X, wherein E is an electrophile.

Embodiment 26 provides a compound of Formula IV, or a salt, solvate, geometric isomer, or stereoisomer thereof:

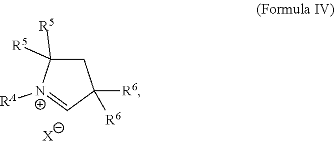

wherein: R^{A}, R⁵ and R⁶ is independently chosen from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl, wherein the optional substitution is by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; X is a counter anion; and R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl.

Embodiment 25 provides a compound of Formula IVa, or a salt, solvate, geometric isomer, or stereoisomer thereof:

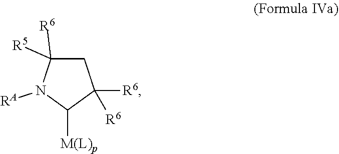

wherein: R^{A}, R⁵ and R⁶ is independently chosen from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl, wherein the optional substitution is by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; M is an element of Group VIII to Group XVI with an atomic weight greater than 25; L is a ligand of M, wherein at each occurrence L can be the same or different; p is whole number from 0 to 5; and R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, and optionally substituted C₁₋₁₀ acyl.

Embodiment 27 provides a compound of Formula V, or a salt, solvate, geometric isomer, or stereoisomer thereof:

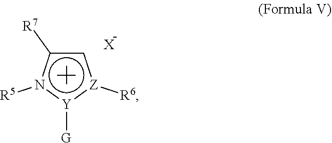

wherein: each R⁵, R⁶ and R′ is independently chosen from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl, wherein the optional substitution is by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; X is a counter anion; Y is N or C; Z is N or C; G is absent, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl, wherein the optional substitution is by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; R is independently at each occurrence hydrogen or C₁₋₁₀ hydrocarbyl, with the proviso that Y and Z are not both C.

Embodiment 28 provides a compound of Formula Va, or a salt, solvate, geometric isomer, or stereoisomer thereof:

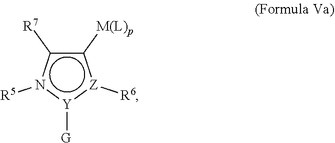

wherein: each R⁵, R⁶ and R⁷ is independently chosen from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl, wherein the optional substitution is by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; Y is N or C; Z is N or C; M is an element of Group VIII to Group XVI with an atomic weight greater than 25; L is a ligand of M, wherein at each occurrence L can be the same or different; p is whole number from 0 to 5; and G is absent, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl, wherein the optional substitution is by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; R is independently at each occurrence hydrogen or C₁₋₁₀ hydrocarbyl, with the proviso that Y and Z are not both C.

1. A compound of Formula I, or a salt, solvate, geometric isomer, or stereoisomer thereof: 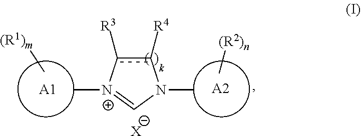
wherein: [symbol]
is a single or double bond;
A1 and A2 are each independently C₆₋₁₈ aryl or C₆₋₁₈ heteroaryl;
R¹ and R² are each independently C₁₋₃ alkyl substituted with at least one aryl or heteroaryl,
wherein the aryl or heteroaryl in R¹ and R² is independently optionally substituted with at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl;
R³ and R⁴ are each independently hydrogen, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl,
wherein the optional substitution independently comprises at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; or
R³ and R⁴ taken together with the ring to which they are attached are used to form a C₄₋₂₀ cycloalkyl, C₆₋₂₀ aryl, or C₆₋₂₀ heteroaryl,
each of which is independently optionally substituted with at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl;
X is a counter anion;
R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl;
k is 1, 2, 3, or 4;
m is an integer from 0 to 6;
n is an integer from 0 to 6; and
with the proviso that if A1 and A2 are phenyl, then at least one of m or n is 3, 4, or 5.

2. The compound of claim 1, wherein at least one of the following applies:
(a) A1 and A2 are identical;
(b) A1 is a C₆₋₁₀ aryl;
(c) A1 is phenyl; and
(d) A1 is naphthyl.

3. (canceled)

4. (canceled)

5. (canceled)

6. The compound of claim 1, wherein at least one of the following applies:
i. m is 3;
ii. n is 3; or
iii. k is 1.

7. The compound of claim 1, wherein the compound is selected from the group consisting of 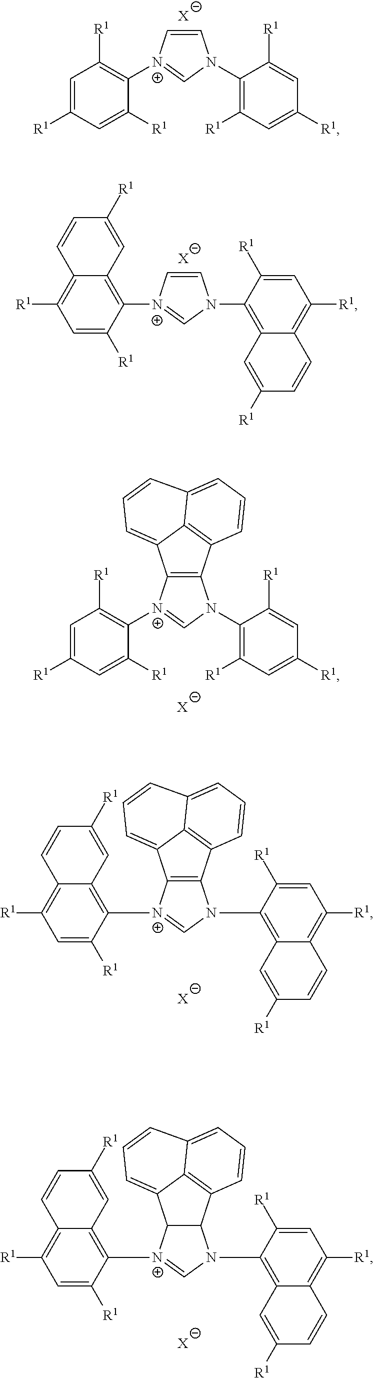 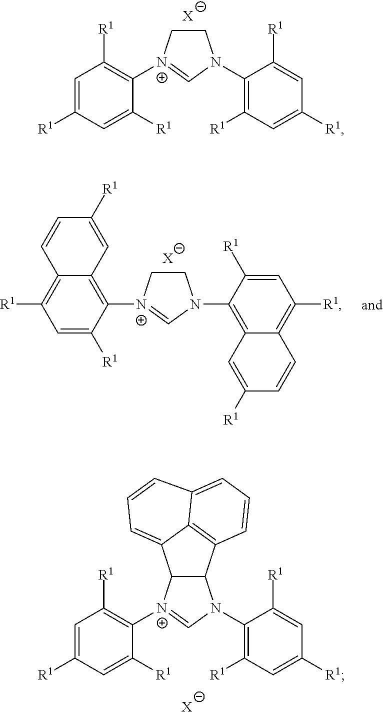
wherein each occurrence of R¹ is independently selected from the group consisting of CH(phenyl)₂, CH(4-Me-C₆H₄), CH(4-t-Bu-C₆H₄)₂, CH(4-MeO—C₆H₄)₂, CH(4-CF₃-C₆H₄)₂, CH(3,5 -dim ethyl-C₆H₃)₂, CH(3,5-ditrifluoromethyl-C₆H₃)₂, and CH(3,5-difluoro-C₆H₃)₂.

8. The compound of claim 7, wherein X is C₁.

9. The compound of claim 1, wherein R¹ and R² are identical.

10. The compound of claim 2, wherein R¹ is CH(aryl)₂.

11. The compound of claim 10, wherein at least one of the following applies:
(a) R¹ is CH(phenyl)₂; and
(b) m is 3 and n is 3.

12. (canceled)

13. The compound of claim 1, wherein X is selected from the group consisting of F, Cl, Br, I, OSO₂R, OSO₃R, and OC(═O)R.

14. The compound of claim 13, wherein X is C₁.

15. A compound of Formula II, or a salt, solvate, geometric isomer, or stereoisomer thereof: 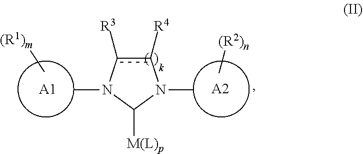
wherein:
M is an element of Group VIII to Group XVI with an atomic weight greater than 25;
L is a ligand of M, wherein at each occurrence L can be the same or different;
p is whole number from 0 to 5; and [symbol]
is a single or double bond;
A1 and A2 are each independently C₆₋₁₀ aryl or C₆₋₁₀ heteroaryl;
R¹ and R² are each independently C₁₋₃ alkyl substituted with at least one aryl or heteroaryl,
wherein the aryl or heteroaryl in R¹ and R² is independently optionally substituted with at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl;
R³ and R⁴ are each independently hydrogen, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, or optionally substituted C₁₋₁₂ acyl,
wherein the optional substitution comprises at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl; or
R³ and R⁴ taken together with the ring to which they are attached are used to form a C₄₋₂₀ cycloalkyl, C₆₋₂₀ aryl, or C₆₋₂₀ heteroaryl,
each of which is independently optionally substituted with at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl;
R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl;
k is 1, 2, 3, or 4;
m is an integer from 0 to 5;
n is an integer from 0 to 5; and
with the proviso that if A1 and A2 are phenyl, then at least one of m or n is 3, 4, or 5.

16. The compound of claim 15, wherein M is selected from the group consisting of Fe, Co, Ni, Cu, Ru, Rh, Pd, Ag, Re, Os, Ir, Pt, and Au.

17. The compound of claim 15, wherein M is a Group XVI element.

18. The compound of claim 15, wherein p is 0, 1, or 2.

19. The compound of claim 15, wherein at least one of the following applies:
(a) L is an anionic ligand; and
(b) L is a neutral ligand.

20. (canceled)

21. The compound of claim 15, wherein at least one L is selected from the group consisting of:
acac, Cl, 3-Cl-pyridine, pyridine, N—R′-imidazole, cinnamyl, allyl, 1-R″-indenyl, Cp (cyclopentadienyl), aniline, 3-CF₃-aniline, μ-OH, 1,4-naphthoquinone, CpCl, PR^{A}₃, di-tert-butyl-fumarate, and methyl methacrylate;
wherein:
R′ is Me, Et, Bu, or Ph;
R″ is t-Bu, i-Pr, Et, Me, Cyclohexyl, or 1-Adamantyl; and
each R^{A} is independently optionally substituted C₁₋₈ alkyl, optionally substituted C₂₋₈ alkenyl, optionally substituted C₂₋₈ alkynyl, optionally substituted C₃₋₈ cycloalkyl, optionally substituted C₁₋₈ heteroalkyl, optionally substituted C₁₋₈ alkoxy, optionally substituted C₆₋₁₀ aryl, or optionally substituted C₄₋₈ heteroaryl.

22. The compound of claim 15, wherein the compound is selected from the group consisting of [Pd(NHC)(acac)Cl], [Pd(NHC)(3-Cl-pyridine)Cl₂], [Pd(NHC)(pyridine)Cl₂], [Pd(NHC)(N—R′-imidazole)Cl₂] [Pd(NHC)(cinnamyl)Cl], [Pd(NHC)(allyl)Cl], [Pd(NHC)(1-R″-indenyl)Cl], [Pd(NHC)(Cp)Cl] (Cp=cyclopentadienyl), [Pd(NHC)(aniline)Cl₂], [Pd(NHC)(3-CF₃-aniline)Cl₂], [Pd(NHC)(μ-Cl)Cl]₂, [Pd(NHC)(μ-OH)Cl]₂, [Pd(NHC)(1,4-naphthoquinone)], 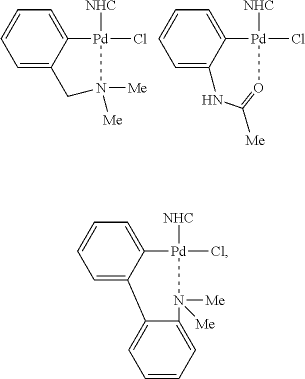
[Ni(NHC)CpCl], [Ni(NHC)(PR^{A}₃)X^{A}₂], [Ni(NHC)(di-tert-butyl-fumarate)₂], [Ni(NHC)(methyl methacrylate)₂], [Au(NHC)X^{B}], [Cu(NHC)X^{A}], [Ag(NHC)X^{A}], 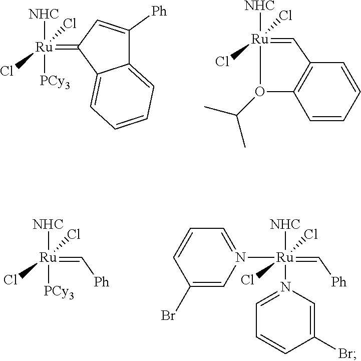
wherein NHC is selected from the group consisting of 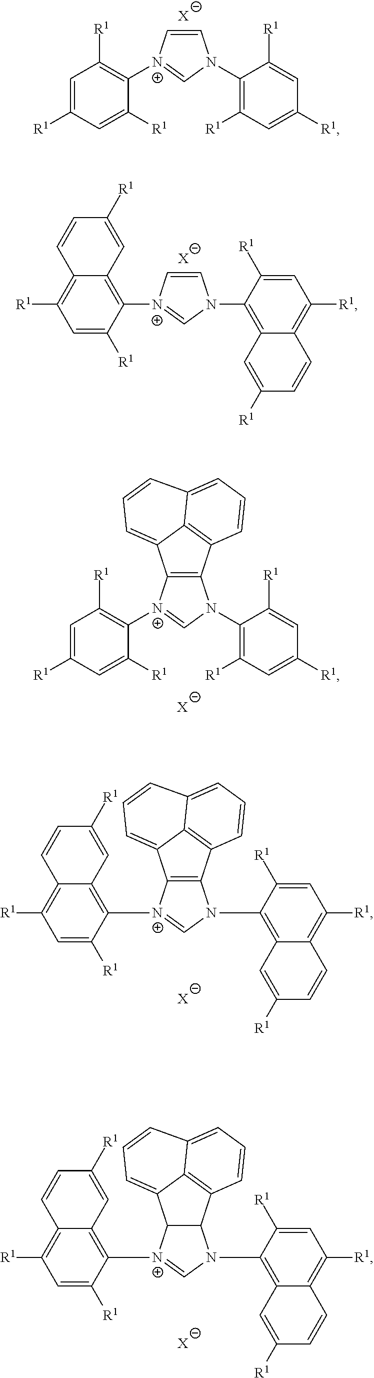 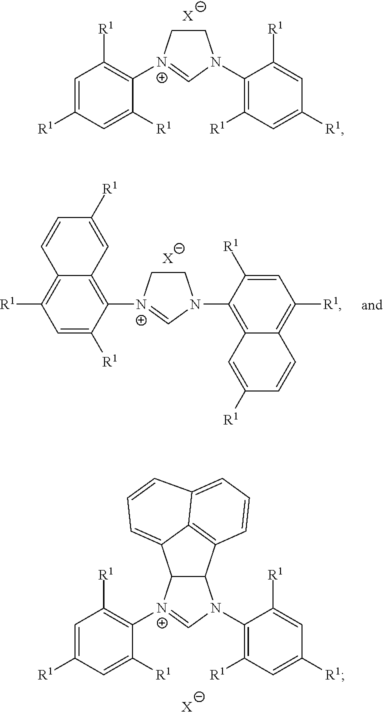
each occurrence of R¹ is independently selected from the group consisting of CH(phenyl)₂, CH(4-Me-C₆H₄), CH(4-t-Bu-C₆H₄)₂, CH(4-MeO—C₆H₄)₂, CH(4-CF₃-C₆H₄)₂, CH(3,5-dimethyl-C₆H₃)₂, CH(3,5-ditrifluoromethyl-C₆H₃)₂, and CH(3,5-difluoro-C₆H₃)₂;
R′ is Me, Et, Bu, or Ph;
R″ is t-Bu, i-Pr, Et, Me, Cyclohexyl, or 1-Adamantyl;
each R^{A} is independently optionally substituted C₁₋₈ alkyl, optionally substituted C₂₋₈ alkenyl, optionally substituted C₂₋₈ alkynyl, optionally substituted C₃₋₈ cycloalkyl, optionally substituted C₁₋₈ heteroalkyl, optionally substituted C₁₋₈ alkoxy, optionally substituted C₆₋₁₀ aryl, or optionally substituted C₄-C₈ heteroaryl;
X is Cl or Br;
X^{A} is Cl, Br, or I; and
X^{B} is Cl, Br, I, NTf₂, or OTf.

23. A method of making the compound of claim 2, the method comprising:
contacting a compound, or a salt, solvate, geometric isomer, or stereoisomer thereof,
having the structure: 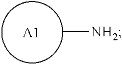
with a cationic form of R¹, to form a compound of Formula III, or a salt, solvate, geometric isomer, or stereoisomer thereof: 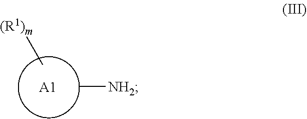
condensing the compound of Formula III with (CHO)₂ to form a diimine compound having the structure 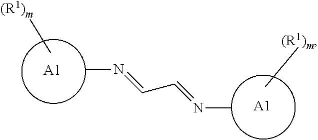
or a salt, solvate, geometric isomer, or
stereoisomer thereof; and
cyclizing the diimine compound to form the compound of Formula I, or a salt, solvate, geometric isomer, or stereoisomer thereof.

24. The method of claim 23, wherein at least one of the following applies:
(a) the cationic form of R¹ is Ph₂CH⁺; and
(b) the cyclizing comprises reacting the diamine compound with E-X, wherein E is an electrophile.

25. (canceled)

26. A compound of Formula IV, or a salt, solvate, geometric isomer, or stereoisomer thereof: 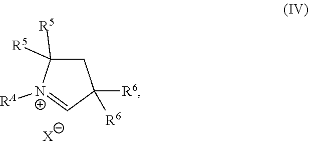
wherein:
each occurrence of R^{A}, R⁵ and R⁶ is independently chosen from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₈ aryl, optionally substituted C₂-C₁₂ heterocyclyl, optionally substituted C₄-C₁₈ heteroaryl, optionally substituted C₁₋₁₂ acyl, and C₁₋₃ alkyl substituted with at least one aryl or heteroaryl,
wherein the optional substitution is independently by at least one group selected from the group consisting of halogen, OR, SiR₃, OSiR₃, OSiR₃, OSi(OR)₃, BR₃, BR₂, B(OR)₃, B(OR)₂, CN, CF₃, OCF₃, SO₂R, SO₂N(R)₂, SO₃R, C(O)R, NR₂, N(R)SO₂R, N(R)SO₂N(R)₂, (CH₂)₀₋₂N(R)C(O)R, (CH₂)₀₋₂N(R)N(R)₂, N(R)C(O)OR, optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted C₁₋₁₂ heteroalkyl, optionally substituted C₁₋₁₂ alkoxy, optionally substituted C₆₋₁₂ aryl, optionally substituted C₂-C₁₂ heterocyclyl, optionally substituted C₄-C₁₂ heteroaryl, and optionally substituted C₁₋₁₂ acyl;
X is a counter anion; and
R is independently at each occurrence hydrogen, optionally substituted C₁₋₁₀ alkyl, optionally substituted C₂₋₁₀ alkenyl, optionally substituted C₂₋₁₀ alkynyl, optionally substituted C₃₋₁₀ cycloalkyl, optionally substituted C₁₋₁₀ heteroalkyl, optionally substituted C₁₋₁₀ alkoxy, optionally substituted C₆₋₁₀ aryl, optionally substituted C₂-C₁₀ heterocyclyl, optionally substituted C₄-C₁₀ heteroaryl, or optionally substituted C₁₋₁₀ acyl.

27-29. (canceled)
